(19) United States
(12) Patent Application Publication
(10) Pub. No.: US 2011/0135588 A1
(43) Pub. Date: Jun. 9, 2011

(54) Hair Conditioning Composition Comprising Quaternized Silicone Polymer, Silicone Copolymer, and Carbamate

(76) Inventors: Nobuaki UEHARA, Kobe (JP); Sang Ja Choi, Osaka (JP)

(21) Appl. No.: 12/962,095

(22) Filed: Dec. 7, 2010

Related U.S. Application Data
(60) Provisional application No. 61/267,481, filed on Dec. 8, 2009.

(51) Int. Cl. A61K 8/89; A61Q 5/12

(52) U.S. Cl. 424/70.12

(57) ABSTRACT

Disclosed is a hair conditioning composition comprising by weight: (a) from about 0.1% to about 15% of a silicone polymer containing quaternary groups wherein said silicone polymer comprises silicone blocks with greater than about 200 siloxane units; (b) a solvent for the silicone polymer which is a silicone copolyol, at a level such that the weight % of the silicone copolyol in its mixture with the quaternized silicone polymer is in the range of from about 1% to about 50%; (c) from about 0.001% to about 0.1% of a carbamate preservative; (d) an aqueous carrier.

### CROSS REFERENCE TO RELATED APPLICATIONS

This application claims the benefit of U.S. Provisional Application No. 61/267,481 filed on Dec. 8, 2009.

### FIELD OF THE INVENTION

The present invention relates to a hair conditioning composition comprising by weight: (a) from about 0.1% to about 15% of a silicone polymer containing quaternary groups wherein said silicone polymer comprises silicone blocks with greater than about 200 siloxane units; (b) a solvent for the silicone polymer which is a silicone copolyol, at a level such that the weight % of the silicone copolyol in its mixture with the quaternized silicone polymer is in the range of from about 1% to about 50%; (c) from about 0.001% to about 0.1% of a carbamate preservative; (d) an aqueous carrier.

### BACKGROUND OF THE INVENTION

Human hair becomes soiled due to its contact with the surrounding environment and from the sebum secreted by the scalp. The soiling of hair causes it to have a dirty feel and an unattractive appearance. The soiling of the hair necessitates shampooing with frequent regularity.

Shampooing cleans the hair by removing excess soil and sebum. However, shampooing can leave the hair in a wet, tangled, and generally unmanageable state. Once the hair dries, it is often left in a dry, rough, lusterless, or frizzy condition due to removal of the hair's natural oils and other natural conditioning and moisturizing components. The hair can further be left with increased levels of static upon drying, which can interfere with combing and result in a condition commonly referred to as “fly-away hair,” or contribute to an undesirable phenomenon of “split ends.” Further, chemical treatments, such as perming, bleaching, or coloring hair, can also damage hair and leave it dry, rough, lusterless, and damaged.

A variety of approaches have been developed to condition the hair. A common method of providing conditioning benefits to the hair is through the use of conditioning agents such as cationic surfactants and polymers, high melting point fatty compounds, low melting point oils, silicone compounds, and mixtures thereof.

However, there still exists a desire for hair conditioning compositions which provide improved conditioning benefits such as smooth feel and reduced friction.

A variety of approaches have been developed to obtain such conditioning benefits. For example, PCT publication WO2006/138201 discloses a hair conditioning composition comprising a silicone polymer containing quaternary groups and a gel matrix, the gel matrix comprising a cationic surfactant, high melting point fatty compound, and an aqueous carrier in Claim 1. PCT publication WO2006/138201 also discloses, in Examples, the hair conditioning compositions further containing SF1488 silicone copolyol, 0.4% of benzyl alcohol, and 0.0005% of Methylchloroisothiazolinone/Methylisothiazolinone.

However, there remains a need for hair conditioning compositions providing improved mildness/gentleness to skin, scalp and/or eye, while providing improved benefits such as reduced friction.

None of the existing art provides all of the advantages and benefits of the present invention.

### SUMMARY OF THE INVENTION

The present invention is directed to a hair conditioning composition comprising by weight:
(a) from about 0.1% to about 15% of a silicone polymer containing quaternary groups wherein said silicone polymer comprises silicone blocks with greater than about 200 siloxane units;
(b) a solvent for the silicone polymer which is a silicone copolyol, at a level such that the weight % of the silicone copolyol in its mixture with the quaternized silicone polymer is in the range of from about 1% to about 50%;
(c) from about 0.001% to about 0.1% of a carbamate preservative;
(d) an aqueous carrier.

There are at least several preservatives such as parabens, diols, salicylic acid, potassium sorbate and carbamates which are known to be mildness/gentleness to skin, scalp and/or eye, compared to Methylisothiazolinone and its combination with Methylchloroisothiazolinone. However, the inventors of the present invention have surprisingly found that, when using parabens, salicylic acid and potassium sorbate together with quaternized silicone polymer and its solvent of the present invention, the quaternized silicone polymers form agglomerates which is not desirable from product appearance standpoint. Such agglomeration may not be desirable in view of delivering the benefit from the quaternized silicone polymers, too. The inventors of the present invention have also surprisingly found that, when using diols together with gel matrix which is one of the preferred embodiments of the present invention, the product rheology is significantly changed. Based on the above findings, the inventors have finally made the composition of the present invention with carbamate preservatives.

There and other features, aspects, and advantages of the present invention will become better understood from a reading of the following description, and appended claims.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

### Carbamate Preservative

The composition of the present invention comprises a carbamate preservative. It is believed that; carbamates provide the composition of the present invention with mildness to skin, scalp and/or eye, while not deteriorating product appearance.

The carbamate preservative is present in an amount of from about 0.001% to about 0.1%, preferably from about 0.008% to about 0.05%, more preferably from about 0.01% to about 0.05%, and still more preferably from about 0.01% to about 0.02%, by weight of the composition.

Such carbamate preservatives useful herein are alkynyl carbamates of formula (I) below:

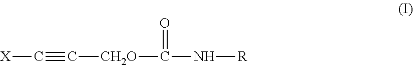

wherein X denotes a halogen atom, and R denotes a hydrogen atom, an alkyl group, or a hydroxyalkyl group. Such halogen atom denotes a fluorine, chlorine, bromine or iodine atom and preferably an iodine atom. Such alkyl group denotes linear or branched groups containing from 1 to 20 carbon atoms, such as, for example, octyl, nonyl, decyl, dodecyl or pentadecyl groups, preferably denotes lower alkyl groups containing from 1 to 4 carbon atoms in a linear or branched chain, such as, for example, methyl, ethyl, propyl, isopropyl, n-propyl, butyl, n-butyl or tert-butyl groups. Among the above alkynyl carbamates, preferred are those of formula (I) in which X denotes an iodine atom and R denotes a lower alkyl group containing from 1 to 4 carbon atoms, in view of preservative efficacy in the composition of the present invention and in view of compatibility with the silicone polymer containing quaternary groups used in the present invention. More preferred is 3-iodo-2-propynyl butylcarbamate

### Other Preservatives

The composition of the present invention can contain paraben preservatives in addition to the carbamate preservatives. In the present invention, the total level of such paraben preservatives, if included, can be 0.35% or less, preferably 0.2% or less, more preferably 0.12% or less, still more preferably below 0.01% (not including 0.01%) by weight of the composition. Preferably, the composition is substantially free of paraben preservatives, in view of not deteriorating conditioning benefits from the silicone compound. In the present invention, “the composition being substantially free of paraben preservatives” means that: the composition is free of paraben preservatives; or, if the composition contains paraben preservatives, the level of such paraben preservatives is very low. Most preferably, the total level of such paraben preservatives is 0% by weight of the composition. Such paraben preservatives include, for example, methylparaben, propylparaben, butylparaben, isopropylparaben.

The composition of the present invention can contain salt preservatives and acid preservatives in addition to the carbamate preservatives. In the present invention, the total level of such salt preservatives and acid preservatives, if included, can be 0.18% or less, preferably 0.15% or less, more preferably 0.1% or less, still more preferably below 0.01% (not including 0.01%) by weight of the composition. Preferably, the composition is substantially free of salt preservatives and acid preservatives, in view of not deteriorating conditioning benefits from the silicone compound. In the present invention, “the composition being substantially free of salt preservatives and acid preservatives” means that: the composition is free of salt preservatives and acid preservatives; or, if the composition contains salt preservatives and/or acid preservatives, the level of such salt preservatives and acid preservatives is very low. Most preferably, the total level of such salt preservatives and acid preservatives is 0% by weight of the composition. Such salt preservatives include, for example, potassium sorbate. Such acid preservatives include, for example, salicylic acid.

Preferably, other preservatives, which can be used in addition to the carbamate preservatives, are not those described above. Such other preservatives are those compatible with ingredients required in the composition of the present invention such as the silicone compound, and other optional ingredients when included such as gel matrix. Such other preservatives include, for example, benzyl alcohol, phenoxy ethanol. Such other preservatives, when included, is present in an amount of preferably from about 0.1% to about 2.5%, more preferably from about 0.2% to about 2.0%, still more preferably from about 0.3% to about 1.5%, and even more preferably from about 0.4% to about 1.0% by weight of the composition.

### Quarternized Silicone Polymer

The compositions of the present invention comprise a silicone polymer containing quaternary groups. The quaternized silicone polymer provides improved conditioning benefits such as smooth feel, reduced friction, prevention of hair damage. Especially, the quaternary group can have good affinity with damaged/colorant hairs. The quaternized silicone polymer is present in an amount of from about 0.1% to about 15%, preferably from about 0.2% to about 10%, more preferably from about 0.3% to about 5%, and even more preferably from about 0.5% to about 4% by weight of the composition.

The silicone polymer of the present invention is comprised of at least one silicone block and at least one non-silicone block containing quaternary nitrogen groups, wherein the number of the non-silicone blocks is one greater than the number of the silicone blocks. The silicone polymers correspond to the general structure (I):

A¹B-(A²-B)ₘ-A¹ (I)

wherein,
B is the silicone blocks with greater than 200 siloxane units;
A² is the non-silicone blocks containing quaternary nitrogen groups;
A¹ are end groups which may contain quaternary groups; and
m is an integer 0 or greater, with the proviso that if m=0 then the A¹ groups contain quaternary groups.

Structures corresponding to the general formula, for example, are disclosed in U.S. Pat. No. 4,833,225, in U.S. Patent Application Publication No. 2004/0138400, and in U.S. Patent Application Publication No. 2004/0048996.

In one embodiment, the silicone polymers can be represented by the following structure (II)

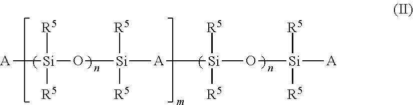

wherein,
A is a group which contains at least one quaternary nitrogen group, and which is linked to the silicon atoms of the silicone block by a silicon-carbon bond, each A independently can be the same or different;
R⁵ is an alkyl group of from about 1 to about 22 carbon atoms or an aryl group; each R⁵ independently can be the same or different;
m is an integer of from 0 or greater, preferably m is less than 20, more preferably m is less than 10; and
n is an integer greater than about 200, preferably greater than about 250, more preferably greater than about 300; preferably less than about 700, more preferably less than about 500.

A preferred structure (III) is with R⁵ as methyl,

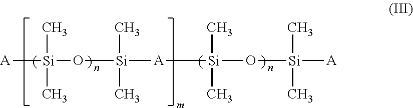

wherein,
A is a group which contains at least one quaternary nitrogen group and is linked to the silicone atoms of the silicone block by a silicon-carbon bond, each A independently can be the same or different;
m is an integer of from 0 or greater, preferably m is less than 20, more preferably m is less than 10;
and n is an integer greater than about 200, preferably greater than about 250, more preferably greater than about 300; preferably less than about 700, more preferably less than about 500.

In another embodiment, the repeat unit of the silicone polymers (the (A²-B) repeat unit in structure (I)) can be represented by the following structure (IV):

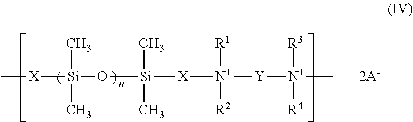

wherein,
X is a bivalent hydrocarbon radical with at least about 4 carbon atoms, which contains a hydroxyl group and can be interrupted by an oxygen atom, and the groups X in the repetition units can be the same or different;
Y is a bivalent hydrocarbon radical with at least about 2 carbon atoms, which can contain a hydroxyl group and which can be interrupted by one or more oxygen or nitrogen atoms, preferably one oxygen atom or one nitrogen atom;
R¹, R², R³, and R⁴ are the same or different and represent a hydrogen or alkyl groups with from about 1 to about 4 carbon atoms or benzyl groups; in one embodiment, the groups R1 and R³, or R² and R⁴ are components of a single alkylene group which connects the two N⁺ atoms;
A⁻ is an inorganic or organic anion;
n is an integer greater than about 200, preferably greater than about 250, more preferably greater than about 300; preferably less than about 700, more preferably less than about 500.

In another embodiment, the A¹-B-(A²-B)ₘ-A¹ silicone block copolymer can be described as a polysiloxane compound containing:

a) at least one polyalkylene oxide structural unit with the general structures (V-VIII):

-A-E-; (V)

-E-A-: (VI)

-A-E-A′-: and/or (VII)

-A′-E-A-. (VIII)

wherein,
A is selected from the group consisting of —CH₂C(O)O—, —CH₂CH₂C(O)O—, —CH₂CH₂CH₂C(O)O—, —OC(O)CH₂—, —OC(O)CH₂—, —OC(O)CH₂CH₂—, and —OC(O)CH₂CH₂CH₂—;
A′ is selected from the group consisting of —CH₂C(O)—, —CH₂CH₂C(O)—, —CH₂CH₂CH₂C(O)—, —C(O)CH₂—, —C(O)CH₂—, —C(O)CH₂CH₂—, and, —C(O)CH₂CH₂CH₂—;
E is a polyalkylene oxide group selected from the group consisting of —[CH₂CH₂O]_{q}—[CH₂CH(CH₃)O]ᵣ—, and, —[OCH(CH₃)CH₂]ᵣ—[OCH₂CH₂]_{q}—; wherein q is from about 1 to about 200; wherein r is from about 0 to about 200.
   wherein,
the terminal position oxygen atom of A binds to the terminal position —CH₂— group of E, and the terminal position carbonyl carbon atom of A′ binds to the terminal position oxygen atom of E forming ester groups in each case, and/or at least one terminal position polyalkylene oxide structural unit of the structure (IX)

-A-E-R² (IX)

wherein,
A and E are the same as above; and
R² is H, straight chain, cyclical or branched C₁ to C₂₀ hydrocarbon group, which can be interrupted by —O—, or —C(O)— and substituted with —OH, and can be acetylene, olefinic, or aromatic;

b) at least one bivalent or trivalent organic group which contains at least one ammonium group;

c) at least one polysiloxane structural unit with the general structure (X)

—K—S—K—, (X)

wherein,
S conforms to the following structure (XI)

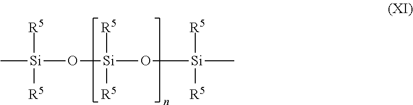

wherein,
R⁵ is an alkyl group of from about 1 to about 22 carbon atoms or an aryl group, and wherein each R⁵ independently can be the same or different,
n is an integer greater than about 200, preferably greater than about 250, more preferably greater than about 300; preferably less than about 700, more preferably less than about 500.
The S groups can be the same or different if several S groups are present in the polysiloxane compound.
K in structure (X) is a bivalent or trivalent straight chain, cyclical, or branched C₂ to C₄₀ hydrocarbon group which is interrupted by —O—, —NH—, —NR⁵—, —C(O)—, —C(S)—,

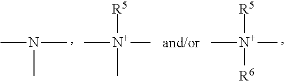

and is substituted by OH; wherein,
R⁵ is as defined above in structure (XI), or represents a bond to a bivalent group R⁶;
   wherein,
R⁶ represents a monovalent or bivalent straight chain, cyclical or branched C₁ to C₂₀ hydrocarbon group which is interrupted by —O—, —NH—, —C(O)—, or —C(S)— and can be substituted with —OH or -A-E-R² wherein A, E, and R² are defined as in structure (IX) above.
The K groups can be identical or different from each other, and in the event K represents a trivalent group, the saturation of the third valence takes place through a bonding to the above mentioned organic group which contains at least one ammonium group;

d) at least one organic or inorganic acid group for neutralization of the charges resulting from the ammonium groups.

A more preferred embodiment is the following structure (XII)

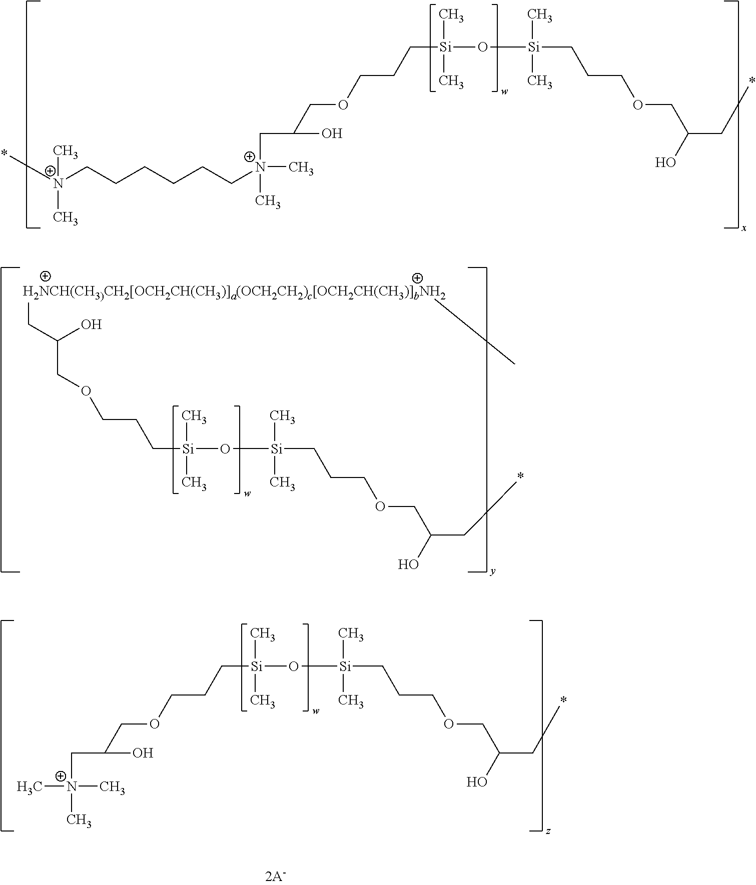

wherein x, y, and z represent mole fractions of the respective components, and hence x+y+z=1;
a+b is less than about 200, preferably a+b is less than about 20, more preferably a+b is less than about 10;
c is less than about 200, preferably c is less than about 100, more preferably c is less than about 50;
w is an integer greater than about 200, preferably greater than about 250, more preferably greater than about 300; preferably less than about 700, more preferably less than about 500; and
A⁻ is an organic or inorganic anion (for example, the 2A⁻ in the above structure can be laurate and acetate in a 1:1 mole ratio).
Preferably, x is greater than 0.6.

Another more preferred embodiment is the following structure (XIII)

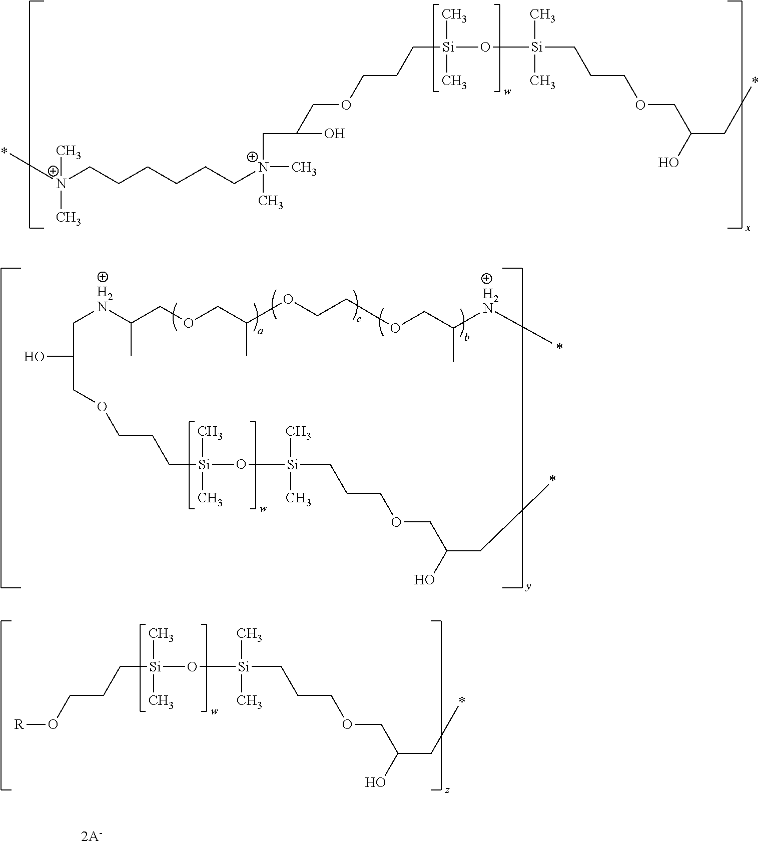

Where R is selected from the groups:

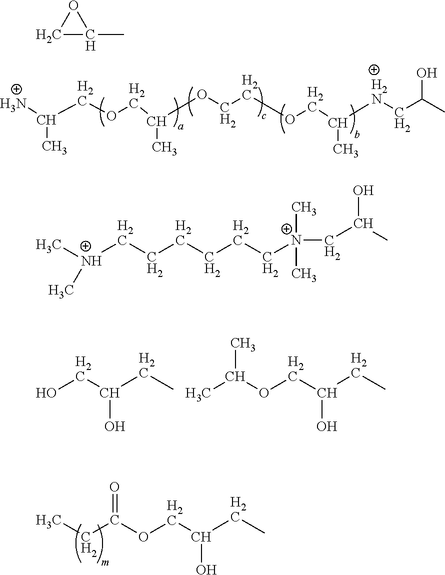

wherein:
x, y, and z represent mole fractions of the respective components, and x+y+z=1;
a+b is an integer from about 2 to about 20;
c is an integer from about 0 to about 200;
w is an integer from about 200 to about 2000, preferably from about 200 to about 800, more preferably from about 250 to about 600 and;
A⁻ is an organic or inorganic anion.
Preferably x is greater than 0.6.

### Silicone Copolyol

The composition of the present invention comprises a silicone copolyol. It is believed that; this silicone copolyol can improve the spreadability of the quaternized silicone polymer, and also can stabilize the quaternized silicone polymer in aqueous conditioner matrix. It is also believed that, by such improved spreadability, the composition of the present invention can provide better dry conditioning benefits such as friction reduction and/or prevention of damage with reduced tacky feel.

The silicone copolyol is contained in the composition at a level such that the weight % of the silicone copolyol to its mixture with quaternized silicone copolymer is in the range of from about 1% to about 50%, preferably from about 5% to about 40%, more preferably from about 10% to 30%.

The silicone copolyols useful herein are those having a silicone backbone such as dimethicone backbone and polyoxyalkylene substitutions such as polyethylene oxide or/and polypropylene oxide substitutions. The silicone copolyol can have such polyoxyalkylene substitutions at the end of the dimethicone backbone. The silicone copolyol can have such polyoxyalkylene substitutions grafted to the dimethicone backbone. The silicone copolyols useful herein have an HLB value of preferably from about 5 to about 17, more preferably from about 8 to about 17, still more preferably from about 8 to about 12. The silicone copolyols having the same INCI name have a variety of HLB value, depending on the molecular weight of the silicone portion and the number of the polyethylene oxide or/and polypropylene oxide substitutions.

Preferred commercially available dimethicone copolyols include, for example: silicone copolyol having a tradename Y-17059 having an HLB value of from about 10 to about 14 (INCI name “Bis-PEG-4 dimethicone”) available from Momemtive; a grafted silicone copolyol having a tradename Silsoft 430 having an HLB value of from about 9 to about 12 (INCI name “PEG/PPG-20/23 dimethicone”) available from GE; a grafted silicone copolyol having a tradename Silsoft 880 having an HLB value of from about 13 to about 17 (INCI name “PEG-12 dimethicone”).

The above quaternized silicone polymer and the silicone copolyol are preferably mixed, prior to incorporating them into a gel matrix formed by cationic surfactants and high melting point fatty compounds. It is believed that, this pre-mixture can improve behavior of the quaternized silicone polymer and the silicone copolyol, for example, increase the stability and spreadability.

When using grafted silicone copolyol, the pre-mixture is preferably further emulsified by a emulsifying surfactant. Such emulsifying surfactant can be used at a level of preferably 0.001% to 1.5%, more preferably 0.005% to 1.0%, still more preferably 0.01% to 0.5% by the weight of the composition. Such surfactants are preferably nonionic, and have an HLB value of preferably from about 2 to about 15, more preferably from about 3 to about 14, still more preferably from about 3 to about 10. Commercially available example of highly preferred emulsifying surfactant is nonionic surfactant having an INCI name C12-C14 Pareth-3 having an HLB value of about 8 supplied from NIKKO Chemicals Co., Ltd. with tradename NIKKOL BT-3.

### Aqueous Carrier

The conditioning composition of the present invention comprises an aqueous carrier. The level and species of the carrier are selected according to the compatibility with other components, and other desired characteristic of the product.

The carrier useful in the present invention includes water and water solutions of lower alkyl alcohols and polyhydric alcohols. The lower alkyl alcohols useful herein are monohydric alcohols having 1 to 6 carbons, more preferably ethanol and isopropanol. The polyhydric alcohols useful herein include propylene glycol, hexylene glycol, glycerin, and propane diol.

Preferably, the aqueous carrier is substantially water. Deionized water is preferably used. Water from natural sources including mineral cations can also be used, depending on the desired characteristic of the product. Generally, the compositions of the present invention comprise from about 20% to about 99%, preferably from about 30% to about 95%, and more preferably from about 80% to about 95% water.

### Gel Matrix

The composition of the present invention may comprises a gel matrix. The gel matrix comprises a cationic surfactant, a high melting point fatty compound, and an aqueous carrier.

The gel matrix is suitable for providing various conditioning benefits such as slippery feel during the application to wet hair and softness and moisturized feel on dry hair. In view of providing the above gel matrix, the cationic surfactant and the high melting point fatty compound are contained at a level such that the weight ratio of the cationic surfactant to the high melting point fatty compound is in the range of, preferably from about 1:1 to about 1:10, more preferably from about 1:1 to about 1:6.

Preferably, when the composition contains gel matrix, the composition is substantially free of anionic surfactants and anionic polymers, in view of stability of the gel matrix. In the present invention, “the composition being substantially free of anionic surfactants and anionic polymers” means that: the composition is free of anionic surfactants and anionic polymers; or, if the composition contains anionic surfactants and anionic polymers, the level of such anionic surfactants and anionic polymers is very low. In the present invention, the total level of such anionic surfactants and anionic polymers, if included, 1% or less, preferably 0.5% or less, more preferably 0.1% or less by weight of the composition. Most preferably, the total level of such anionic surfactants and anionic polymers is 0% by weight of the composition.

It is also preferred, when the composition contains gel matrix, the composition is substantially free of diol preservatives, in view of rheology stability of the gel matrix. In the present invention, “the composition being substantially free of diol preservatives” means that: the composition is free of diol preservatives; or, if the composition contains diol preservatives, the level of such diol preservatives is very low. In the present invention, the total level of such diol preservatives, if included 0.18% or less, preferably 0.15% or less, more preferably 0.1% or less by weight of the composition. Most preferably, the total level of such diol preservatives is 0% by weight of the composition.

Such diol preservatives include, for example, 1,2-decane diol, 1,2-octane diol, 1,2-hexanediol.

### Cationic Surfactant

The composition of the present invention may contain a cationic surfactant, especially for forming a gel matrix described above in detail. The cationic surfactant can be included in the composition at a level from about 1%, preferably from about 1.5%, more preferably from about 1.8%, still more preferably from about 2.0%, and to about 8%, preferably to about 5%, more preferably to about 4% by weight of the composition.

A variety of cationic surfactants including mono- and di-alkyl chain cationic surfactants can be used in the compositions of the present invention. Among them, preferred are mono-alkyl chain cationic surfactants in view of providing desired gel matrix and wet conditioning benefits. The mono-alkyl cationic surfactants are those having one long alkyl chain which has from 12 to 22 carbon atoms, preferably from 16 to 22 carbon atoms, more preferably C18-22 alkyl group, in view of providing balanced wet conditioning benefits. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms. Such mono-alkyl cationic surfactants include, for example, mono-alkyl quaternary ammonium salts and mono-alkyl amines. Mono-alkyl quaternary ammonium salts include, for example, those having a non-functionalized long alkyl chain. Mono-alkyl amines include, for example, mono-alkyl amidoamines and salts thereof. Di-alkyl cationic surfactants herein are those having two long alkyl chains of from 12 to 22 carbon atoms, including, for example, di-long alkyl quaternized ammonium salts.

Mono-Long alkyl Quaternized Ammonium Salt Cationic Surfactant

One of the preferred cationic surfactants of the present invention is a salt of a mono-long alkyl quaternized ammonium and an anion, wherein the anion is selected from the group consisting of halides such as chloride and bromide, C1-C4 alkyl sulfate such as methosulfate and ethosulfate, and mixtures thereof.

The mono-long alkyl quaternized ammonium salts useful herein are those having the formula (I):

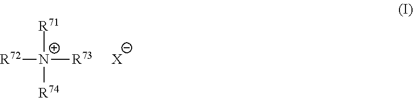

wherein one of R⁷¹, R⁷², R⁷³ and R⁷⁴ is selected from an aliphatic group of from 16 to 40 carbon atoms or an aromatic, alkoxy, polyoxyalkylene, alkylamido, hydroxyalkyl, aryl or alkylaryl group having up to about 40 carbon atoms; the remainder of R⁷¹, R⁷², R⁷³ and R⁷⁴ are independently selected from an aliphatic group of from 1 to about 8 carbon atoms or an aromatic, alkoxy, polyoxyalkylene, alkylamido, hydroxyalkyl, aryl or alkylaryl group having up to about 8 carbon atoms; and X⁻ is a salt-forming anion selected from the group consisting of halides such as chloride and bromide, C1-C4 alkyl sulfate such as methosulfate and ethosulfate, and mixtures thereof. The aliphatic groups can contain, in addition to carbon and hydrogen atoms, ether linkages, and other groups such as amino groups. The longer chain aliphatic groups, e.g., those of about 16 carbons, or higher, can be saturated or unsaturated. Preferably, one of R⁷¹, R⁷², R⁷³ and R⁷⁴ is selected from an alkyl group of from 16 to 40 carbon atoms, more preferably from 18 to 26 carbon atoms, still more preferably from 22 carbon atoms; and the remainder of R⁷¹, R⁷², R⁷³ and R⁷⁴ are independently selected from CH₃, C₂H₅, C₂H₄OH, CH₂C₆H₅, and mixtures thereof. It is believed that such mono-long alkyl quaternized ammonium salts can provide improved slippery and slick feel on wet hair, compared to multi-long alkyl quaternized ammonium salts. It is also believed that mono-long alkyl quaternized ammonium salts can provide improved hydrophobicity and smooth feel on dry hair, compared to amine or amine salt cationic surfactants.

Among them, more preferred cationic surfactants are those having a longer alkyl group, i.e., C18-22 alkyl group. Such cationic surfactants include, for example, behenyl trimethyl ammonium chloride, methyl sulfate or ethyl sulfate, and stearyl trimethyl ammonium chloride, methyl sulfate or ethyl sulfate. Further preferred are behenyl trimethyl ammonium chloride, methyl sulfate or ethyl sulfate, and still further preferred is behenyl trimethyl ammonium chloride. It is believed that; cationic surfactants having a longer alkyl group provide improved deposition on the hair, thus can provide improved conditioning benefits such as improved softness on dry hair, compared to cationic surfactant having a shorter alkyl group. It is also believed that such cationic surfactants can provide reduced irritation, compared to cationic surfactants having a shorter alkyl group.

### Mono-Long Alkyl Amine Cationic Surfactant

Mono-alkyl amines are also suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines in the present invention are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid. The amines herein are preferably partially neutralized with any of the acids at a molar ratio of the amine to the acid of from about 1:0.3 to about 1:2, more preferably from about 1:0.4 to about 1:1.

### High Melting Point Fatty Compound

The composition of the present invention may contain a high melting point fatty compound, especially for forming a gel matrix described above in detail. The high melting point fatty compound can be included in the composition at a level of from about 1% to about 20%, preferably from about 3% to about 10%, more preferably from about 4% to about 8% by weight of the composition.

The high melting point fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are preferably used in the composition of the present invention. The fatty alcohols useful herein are those having from about 14 to about 30 carbon atoms, preferably from about 16 to about 22 carbon atoms. These fatty alcohols are saturated and can be straight or branched chain alcohols. Preferred fatty alcohols include, for example, cetyl alcohol, stearyl alcohol, behenyl alcohol, and mixtures thereof.

High melting point fatty compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

### Silicone Copolymer Emulsion

Compositions of the present invention also comprise a silicone copolymer emulsion with an internal phase viscosity of greater than about 100×10⁶ mm²/s, in view of providing clean feel. It is also believed that this silicone copolymer emulsion can provide body/fullness to hair. The silicone copolymer emulsion is present in an amount of from about 0.1% to about 15%, preferably from about 0.3% to about 10%, and more preferably from about 0.5% to about 5%, by weight of the composition. When included, it is also preferred that the silicone copolymer emulsion is present at a level such that the wt. % of the silicone copolymer emulsion in its mixture with the quaternized silicone polymer and the silicone copolyol is in the range of from about 10% to 300%, more preferably from about 20% to about 200%, still more preferably from about 40% to about 400%.

The silicone copolymer emulsion has a viscosity at 25° C. of greater than about 100×10⁶ mm²/s, preferably greater than about 120×10⁶ mm²/s, more preferably greater than about 150×10⁶ mm²/s. The silicone copolymer emulsion has a viscosity at 25° C. of, preferably less than about 1000×10⁶ mm²/s, more preferably less than about 500×10⁶ mm²/s, and even more preferably less than about 300×10⁶ mm²/s. To measure the internal phase viscosity of the silicone copolymer emulsion, one must first break the polymer from the emulsion. By way of example, the following procedure can be used to break the polymer from the emulsion: 1) add 10 grams of an emulsion sample to 15 milliliters of isopropyl alcohol; 2) mix well with a spatula; 3) decant the isopropyl alcohol; 4) add 10 milliliters of acetone and knead polymer with spatula; 5) decant the acetone; 6) place polymer in an aluminum container and flatten/dry with a paper towel; and 7) dry for two hours in an 80° C. The polymer can then be tested using any known rheometer, such as, for example, a CarriMed, Haake, or Monsanto rheometer, which operates in the dynamic shear mode. The internal phase viscosity values can be obtained by recording the dynamic viscosity (n′) at a 9.900*10⁻³ Hz frequency point. The average particle size of the emulsions is preferably less than about 1 micron, more preferably less than about 0.7 micron.

The silicone copolymer emulsions of the present invention comprise a silicone copolymer, at least one surfactant, and water.

The silicone copolymer results from the addition reaction of the following two materials in the presence of a metal containing catalyst:

(a) a polysiloxane with reactive groups on both termini, represented by formula (III)

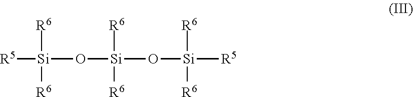

wherein:

R⁵ is a group capable of reacting by chain addition reaction such as, for example, a hydrogen atom, an aliphatic group with ethylenic unsaturation (i.e., vinyl, allyl, or hexenyl), a hydroxyl group, an alkoxyl group (i.e., methoxy, ethoxy, or propoxy), an acetoxyl group, or an amino or alkylamino group; preferably, R⁵ is hydrogen or an aliphatic group with ethylenic unsaturation; more preferably, R⁵ is hydrogen;

R⁶ is alkyl, cycloalkyl, aryl, or alkylaryl and may include additional functional groups such as ethers, hydroxyls, amines, carboxyls, thiols esters, and sulfonates; preferably, R⁶ is methyl. Optionally, a small mole percentage of the groups may be reactive groups as described above for R⁵, to produce a polymer which is substantially linear but with a small amount of branching. In this case, preferably the level of R⁶ groups equivalent to R⁵ groups is less than about 10% on a mole percentage basis, and more preferably less than about 2%;

n is a whole number such that the polysiloxane of formula (III) has a viscosity of from about 1 mm²/s to about 1×10⁶ mm²/s;

and,

(b) at least one silicone compound or non-silicone compound comprising at least one or at most two groups capable of reacting with the R₅ groups of the polysiloxane in formula (III); preferably, the reactive group is an aliphatic group with ethylenic unsaturation.

The metal containing catalysts used in the above described reactions are often specific to the particular reaction. Such catalysts are known in the art. Generally, they are materials containing metals such as platinum, rhodium, tin, titanium, copper, lead, etc.

The mixture used to form the emulsion also contains at least one surfactant. This can include non-ionic surfactants, cationic surfactants, anionic surfactants, alkylpolysaccharides, amphoteric surfactants, and the like. The above surfactants can be used individually or in combination.

An exemplary method of making the silicone copolymer emulsions described herein comprises the steps of 1) mixing materials (a) described above with material (b) described above, followed by mixing in an appropriate metal containing catalyst, such that material (b) is capable of reacting with material (a) in the presence of the metal containing catalyst; 2) further mixing in at least one surfactant and water; and 3) emulsifying the mixture. Methods of making such silicone copolymer emulsions are disclosed in U.S. Pat. No. 6,013,682; PCT Application No. WO01/58986 A1; and European Patent Application No. EP0874017 A2.

Commercially available example of highly preferred silicone copolymer emulsion is an emulsion of about 60-70% of divinyldimethicone/dimethicone copolymer having an internal phase viscosity of minimum 120×10⁶ mm²/s, available from Dow Corning with a tradename HMW2220.

### Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: low melting point oils having a melting point of less than 25° C. including, for example, unsaturated fatty alcohols such as oleyl alcohol and ester oils such as pentaerythritol ester oils; cationic conditioning polymers including, for example, cationic celluloses and cationic guar gums; polyethylene glycols; other conditioning agents such as hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, plant extracts, and nutrients; pH adjusting agents; coloring agents, such as any of the FD&C or D&C dyes; perfumes; and sequestering agents, such as disodium ethylenediamine tetra-acetate; ultraviolet and infrared screening and absorbing agents such as octyl salicylate; and antidandruff agents such as zinc pyrithione.

### Product Forms

The hair care compositions can be formulated into a variety of product forms, including shampoos, conditioners (both rinse-off and leave-on versions), styling products, and the like. In one embodiment, the hair care compositions include only hair conditioners and do not include shampoos or styling products.

The conditioning compositions of the present invention can be in the form of rinse-off products or leave-on products, and can be formulated in a wide variety of product forms, including but not limited to creams, gels, emulsions, mousses and sprays.

The conditioning composition of the present invention is especially suitable for rinse-off hair conditioner. Such compositions are preferably used by following steps:
(i) after shampooing hair, applying to the hair an effective amount of the conditioning compositions for conditioning the hair; and
(ii) then rinsing the hair.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

[Compositions]

| Components | Ex.1 | Ex.2 | Ex.3 | Ex.4 | Ex.5 | Ex.6 | Ex.7 | Ex.8 | Ex. 9 |
|---|---|---|---|---|---|---|---|---|---|
| Quaternized silicone polymer-1 *1 | 1.60 | 1.60 | 1.60 | 1.20 | — | 1.36 | 0.36 | 5.00 | 1.20 |
| Quaternized silicone polymer-2 *2 | — | — | — | — | 1.60 | — | — | — | — |
| Nonionic surfactant *3 | — | — | 0.04 | — | — | — | — | — | — |
| Silicone copolyol-1 *4 | — | — | 0.36 | — | — | — | — | — | — |
| Silicone copolyol-2 *5 | — | — | — | 0.30 | — | — | — | — | — |
| Silicone copolyol-3 *6 | 0.40 | 0.40 | — | — | 0.40 | 0.34 | 0.09 | 1.25 | 0.30 |
| Silicone copolymer | 1.64 | 1.64 | 1.64 | 1.23 | 1.64 | 1.30 | 0.37 | 5.13 | 1.23 |
| emulsion *7 | | | | | | | | | |
| Behenyl trimethyl | 2.28 | — | 2.28 | — | 2.28 | 2.28 | 1.37 | 2.28 | — |
| ammonium chloride | | | | | | | | | |
| Behentrimonium Methosulfate | — | 1.80 | — | 1.80 | — | — | — | — | 1.90 |
| Varisoft 432-PPG *8 | — | — | — | — | — | — | — | — | 0.80 |
| Isopropyl alcohol | 0.57 | 0.45 | 0.57 | 0.45 | 0.57 | 0.57 | 0.34 | 0.57 | 0.47 |
| Cetyl alcohol | 1.86 | 1.49 | 1.86 | 1.86 | 1.86 | 1.86 | 1.12 | 1.86 | 1.48 |
| Stearyl alcohol | 4.64 | 3.71 | 4.64 | 4.64 | 4.64 | 4.64 | 2.78 | 4.64 | 3.71 |
| 3-Iodo-2-propynyl | 0.015 | 0.015 | 0.015 | 0.015 | 0.015 | 0.015 | 0.015 | 0.015 | 0.015 |
| butylcarbamate *9 | | | | | | | | | |
| Benzyl alcohol | 0.50 | 0.50 | 0.50 | 0.50 | 0.50 | 0.50 | 0.50 | 0.50 | 0.50 |
| Phenoxy ethanol | 0.50 | 0.50 | 0.50 | 0.50 | 0.50 | 0.50 | 0.50 | 0.50 | 0.50 |
| Disodium EDTA | 0.013 | 0.013 | 0.013 | 0.013 | 0.013 | 0.013 | 0.013 | 0.013 | 0.013 |
| Perfume | 0.4 | 0.4 | 0.4 | 0.4 | 0.4 | 0.4 | 0.4 | 0.4 | 0.4 |
| Panthenol | 0.05 | 0.05 | 0.05 | 0.05 | 0.05 | 0.05 | 0.05 | 0.05 | 0.05 |
| Panthenyl ethylether | 0.03 | 0.03 | 0.03 | 0.03 | 0.03 | 0.03 | 0.03 | 0.03 | 0.03 |

| Deionized water | q.s. to 100% | | | | | | |
|---|---|---|---|---|---|---|---|
| Components | Ex.10 | Ex.11 | Ex.12 | Ex.13 | Ex.14 | Ex.15 | Ex.16 |
| Quaternized silicone polymer-1 *1 | 1.36 | 1.36 | 1.36 | 1.36 | 1.36 | 1.60 | 1.60 |
| Silicone copolyol-3 *6 | 0.34 | 0.34 | 0.34 | 0.34 | 0.34 | 0.40 | 0.40 |
| Silicone copolymer emulsion *7 | 1.30 | 1.30 | 1.30 | 1.30 | 1.30 | 1.64 | 1.64 |
| Behenyl trimethyl | 2.28 | 2.28 | 2.28 | 2.28 | 2.28 | 2.28 | 2.28 |
| ammonium chloride | | | | | | | |
| Isopropyl alcohol | 0.57 | 0.57 | 0.57 | 0.57 | 0.57 | 0.57 | 0.57 |
| Cetyl alcohol | 1.86 | 1.86 | 1.86 | 1.86 | 1.86 | 1.67 | 1.86 |
| Stearyl alcohol | 4.64 | 4.64 | 4.64 | 4.64 | 4.64 | 4.18 | 4.64 |
| Methylparaben/Propylparaben | — | — | 0.40 | — | — | — | — |
| Methylchloroisothiazolinone/ | 0.033 | — | — | — | — | — | — |
| Methylisothiazolinone *10 | | | | | | | |
| 1,2-Decane diol *11 | — | — | — | 0.20 | — | — | — |
| 1,2-Octane diol/1,2-Hexanediol *12 | — | — | — | — | 1.00 | — | — |
| Methylisothiazolinone *13 | — | 0.093 | — | — | — | — | — |
| Potassium sorbate | — | — | — | — | — | 0.25 | — |
| Salicylic acid | — | — | — | — | — | — | 0.20 |
| Benzyl alcohol | 0.40 | 0.40 | 0.50 | 0.50 | 0.50 | 0.50 | 0.50 |
| Phenoxy ethanol | — | — | 0.50 | 0.50 | 0.50 | 0.50 | 0.50 |
| Disodium EDTA | 0.013 | 0.013 | 0.013 | 0.013 | 0.013 | 0.013 | 0.013 |
| Perfume | 0.4 | 0.4 | 0.35 | 0.25 | 0.25 | 0.4 | 0.4 |
| Panthenol | 0.05 | 0.05 | 0.05 | 0.05 | 0.05 | 0.05 | 0.05 |
| Panthenyl ethylether | 0.05 | 0.03 | 0.03 | 0.05 | 0.05 | 0.03 | 0.03 |

| | |
|---|---|
| Deionized water | q.s. to 100% |

Definitions of Components
*1 Quaternized silicone polymer-1: having the structure (XII), with x = 0.8, y = 0.1, z = 0.1, a + b = 6, c = 39, and w = 350, neat
*2 Quaternized silicone polymer-2: having the structure (XIII), with x = 0.8, y + z = 0.2, a + b = 6, c = 39, and w = 350, neat
*3 Nonionic surfactant: NIKKOL BT-3 available from NIKKO Chemical
*4 Silicone copolyol-1: Silsoft 430 available from GE Silicone
*5 Silicone copolyol-2: Silsoft 880 available from GE Silicone
*6 Silicone copolyol-3: Y-17059 available from Momentive
*7 Silicone copolymer emulsion-1: an emulsion of about 60-70% of divinyldimethicone/dimethicone copolymer having an internal phase viscosity of minimum 120 × 10⁶ mm²/s, available from Dow Corning with a tradename HMW2220
*8 67-69% of Dicetyldimonium Chloride in Propylene Glycol, available from Evonik Goldschmidt Corporation
*9 3-iodo-2-propynyl butylcarbamate: Glycacil available from Lonza
*10 Methylchloroisothiazolinone/Methylisothiazolinone: Kathon CG available from Rohm & Haas
*11 1,2-Decane diol: SymClariol available fromSymrise
*12 1,2-Octane diol/1,2-Hexanediol: Symdiol68 available fromSymrise
*13 Methylisothiazolinone: Neolone950 available from Rohm & Haas

### Method of Preparation

The conditioning compositions of “Ex. 1” through “Ex. 16” as shown above can be prepared by any conventional method well known in the art. They are suitably made as follows:

Cationic surfactants and high melting point fatty compounds are added to water with agitation, and heated to about 80° C. The mixture is cooled down to about 55° C. Quaternized silicone polymers, silicone copolyols, and if included, nonionic surfactants are mixed to form silicone pre-mixture. This silicone pre-mixture is added with agitation to the above mixture of cationic surfactant, high melting fatty compounds and water. If included, other components such as perfumes and preservatives are added to the mixture with agitation. Then the mixture is cooled down to room temperature.

### Properties and Benefits

With respect to the above compositions of Ex. 6, and Ex. 10-16, product properties and preservative alone are evaluated by the following methods. Results of the evaluation are also shown in below Table 1.

Product Appearance (Compatibility with the Quaternized Silicone Polymer and/or Solvent Thereof)

Product appearance is evaluated as follows, by silicone particle size in compositions which was observed using micro scope.

Pass: Average silicone particle size is 200 μm or less

Fail: Average silicone particle size is above 200 μm (excluding 200 μm)

Results of the evaluation are shown below in Table 1.

### Product Rheology

Product rheology is evaluated as follows:

Rheology measurement is done on compositions after being kept at 40° C. for 6 week. Shear stress is measured by shear rate sweep condition with a rheometer available from TA Instruments with a mode name of ARG2. Geometry has 40 mm diameter, 2° C. cone angle, and gap of 49 μm. Shear rate is logarithmically increased from 0 to 1200/s for 1 min, and temperature is kept at 26.7° C. As rheology, share stress at 950/s share rate is evaluated.

A: Control or Equal to control (below 15% increase or decrease of product rheology)

B: From 15% to below 30% increase or decrease of product rheology, compared to Control

C: From 30% to below 45% increase or decrease of product rheology, compared to Control

The composition of Ex. 10, used herein as Control, has an adequate product rheology as a commercial product. Results of the evaluation are shown below in Table 1.

### Preservative Efficacy

Preservative efficacy was confirmed by microbial challenge test. Compositions were tested under simulated consumer use conditions (including diluted conditions) using pools of representative microorganisms that include Pseudomonas, Enterobacteria, mold and yeast species. Survival of the microorganisms was measured at different time points ranging from 0 to 4 weeks.

Pass: Within 4 weeks, survival of the microorganisms becomes below 1.0 cfu/g product (excluding 1.0 cfu/g product).

Fail: After 4 weeks, survival of the microorganisms is still 1 cfu/g product or more.

Results of the evaluation are shown below in Table 1.

TABLE 1

| | Ex.6 | Ex.10 | Ex.12 | Ex.13 | Ex.14 | Ex.15 | Ex.16 |
|---|---|---|---|---|---|---|---|
| Product appearance | Pass | Pass | Fail | Pass | Pass | Fail | Fail |
| Product rheology | A | A | — | B | C | — | — |
| Preservative efficacy | Pass | Pass | — | Pass | Pass | — | — |

Examples 1 through 9 are hair conditioning compositions of the present invention which are particularly useful for rinse-off use. The embodiments disclosed and represented by the previous “Ex. 1” through “Ex. 9” have many advantages. For example, they provide mildness to skin, scalp and/or eye, while not deteriorating product appearance. They also provide such benefits while not deteriorating product rheology.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the same term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair conditioning composition comprising by weight:
(a) from about 0.1% to about 15% of a silicone polymer containing quaternary groups wherein said silicone polymer comprises silicone blocks with greater than about 200 siloxane units;
(b) a solvent for the silicone polymer which is a silicone copolyol, at a level such that the weight % of the silicone copolyol in its mixture with the quaternized silicone polymer is in the range of from about 1% to about 50%;
(c) from about 0.001% to about 0.1% of a carbamate preservative;
(d) an aqueous carrier.

2. The hair conditioning composition of claim 1 comprising from about 0.01% to about 0.02% of a carbamate preservative.

3. The hair conditioning composition of claim 1, wherein the carbamate preservatives are alkynyl carbamates of formula (I): 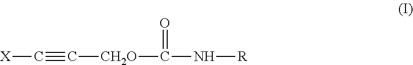
wherein X denotes a halogen atom, and R denotes a hydrogen atom, an alkyl group, or a hydroxyalkyl group.

4. The hair conditioning composition of claim 3, wherein the carbamate preservatives are the alkynyl carbamates of formula (I), wherein X denotes an iodine atom and R denotes a lower alkyl group containing from 1 to 4 carbon atoms.

5. The hair conditioning composition of claim 4, wherein the carbamate preservative is 3-iodo-2-propynyl butylcarbamate.

6. The hair conditioning composition of claim 1, wherein the composition is substantially free of paraben preservatives.

7. The hair conditioning composition of claim 1, wherein the composition is substantially free of salt preservatives and acid preservatives

8. The hair conditioning composition of claim 1, wherein said quaternized silicone polymer corresponds to the following formula:
A¹-B-(A²-B)ₘ-A¹
wherein,
B is a silicone block with greater than about 200 siloxane units;
A² is a non-silicone block containing quaternary nitrogen groups;
A¹ is an end group; and
m is an integer 0 or greater; and
wherein when m is 2 or greater, each A² independently can be the same or different.

9. The hair conditioning composition of claim 1, wherein the quaternized silicone polymer has the following structure (XIII) 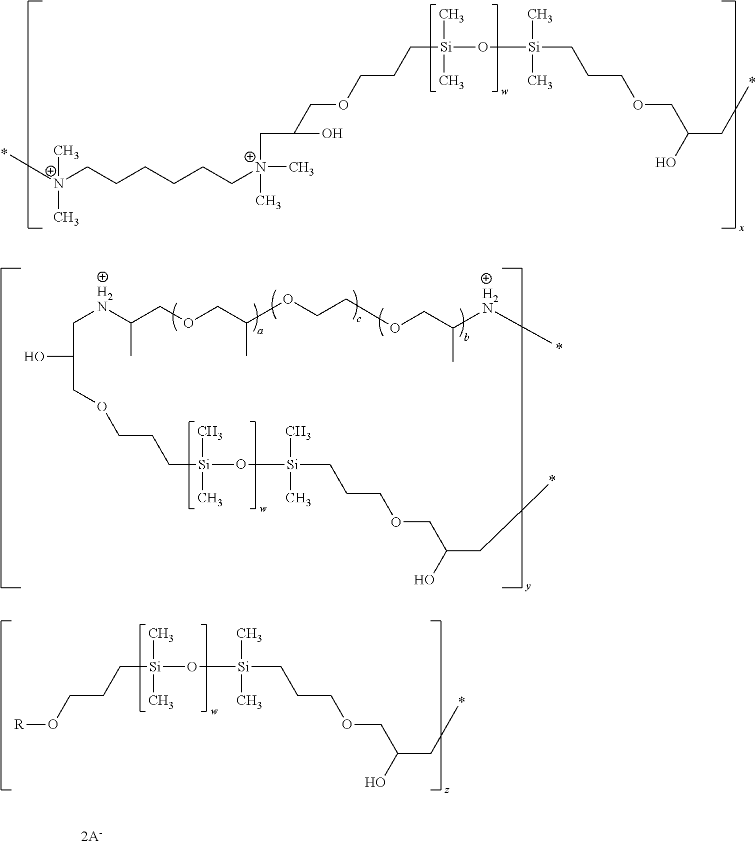
Where R is selected from the groups: 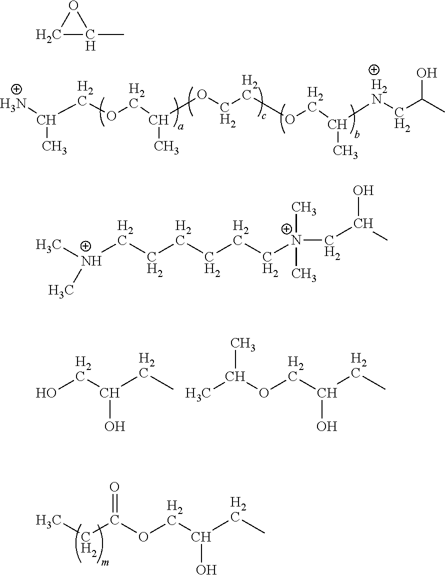
wherein:
x, y, and z represent mole fractions of the respective components, x is greater than 0.6 and x+y+z=1;
a+b is an integer from about 2 to about 20;
c is an integer from about 0 to about 200;
w is an integer from about 200 to about 2000; and
A⁻ is an organic or inorganic anion.

10. The hair conditioning composition of claim 1 further comprising:
(e) from about 0.1% to about 10% by weight of the composition of a cationic surfactant; and
(f) from about 1% to about 20% by weight of the composition of a high melting point fatty compound;
wherein the cationic surfactant, the high melting point fatty compound, and the aqueous carrier form a gel matrix.

11. The hair conditioning composition of claim 10, wherein the composition is substantially free of diol preservatives.

12. The hair conditioning composition of claim 10, wherein the composition is substantially free of anionic surfactants and anionic polymers.
